(19) United States
(12) Patent Application Publication
(10) Pub. No.: US 2004/0097567 A1
(43) Pub. Date: May 20, 2004

(54) Pharmaceutical composition

(76) Inventors: Gerard Moinet, Orsay (FR); Gerard Botton, Buc (FR); Didier Mesangau, Combs La Ville (FR)

Correspondence Address: Millen White Zelano & Branigan, Arlington Court Plaza I, Suite 1400, 2200 Clarendon Boulevard, Arlington, VA, 22201, US

(21) Appl. No.: 10/472,081

(22) PCT Filed: Mar. 8, 2002
(86) PCT No.: PCT/EP02/02561

(30) Foreign Application Priority Data
Mar. 19, 2001 (FR) 0103704

(51) Int. Cl. A61K 31/426; A61K 31/4439

(52) U.S. Cl. 514/369; 514/342

(57) ABSTRACT

The invention relates to a pharmaceutical compositions for the treatment of diabetes comprising, as active principles, a derivation of the 5-phenoxyalkyl-2,4-thiazolidinedione type and a compound that stimulates insulin secretion.

The present invention relates to a pharmaceutical composition comprising, as active principles, a derivative of the 5-phenoxyalkyl-2,4-thiazolidinedione type described in WO 97/47612 and a compound that stimulates insulin secretion.

The invention also relates to the use of a derivative of the 5-phenoxyalkyl-2,4-thiazolidinedione type and a compound that stimulates insulin secretion for the preparation of a medicinal preparation for reducing hyperglycaemia, more particularly non-insulin-dependent hyperglycaemia.

Many thiazolidine-2,4-dione derivatives have been described as anti-hyperglycaemiants and hypolipaemiants and have thus been described as anti-diabetic agents (Takeda, patent EP 193 256 and Sankyo patent EP 207 581). These compounds are activators of the peroxisome proliferator-activated receptor γ (PPARγ).

The compounds that stimulate insulin secretion by the pancreatic β cells are more specifically compounds that act on specific receptors located especially at the surface of the pancreatic β cells.

Thus, sulphonylureas are compounds that stimulate insulin secretion by the pancreatic β cells. These derivatives are hypoglycaemiant active agents. Mention may be made especially of glipizide, gliclazide, glimepiride, glibenclamide, glibornuride, chlorpropamide, tolazamide, tolbutamide and tolcyclamide.

The combination of certain thiazolidine-2,4-dione derivatives, such as pioglitazone and troglitazone, and of compounds that stimulate insulin secretion, more particularly sulphonylureas, has already been described for the treatment of diabetes (Takeda, patent application EP 749 751 and SmithKline Beecham, patent applications WO 98/57649 and WO 99/03476).

Diabetes is a chronic disease that has various pathological manifestations. It is accompanied by disorders of lipid and sugar metabolism and circulatory disorders. In many cases, diabetes tends to progress to a variety of pathological complications. Thus, it is necessary to find the treatment that is suited to each individual suffering from diabetes.

The specific combination of a compound that stimulates insulin secretion with a 5-phenoxyalkyl-2,4-thiazolidinedione that has no activity on the transactivation of PPARγ has not been described and offers particular advantages, especially the absence of weight gain and/or of haemodilution.

Thus, one aim of the present invention is to propose a composition for significantly improving the use of glucose.

A further aim of the invention is to propose a composition that is suitable for treating diabetes by displaying considerable action on the metabolic syndrome of insulin resistance.

A final aim of the invention is to propose a composition that is particularly suitable for diabetics at the various stages of the disease.

These aims and others are achieved by the present invention, which relates to a pharmaceutical composition comprising, as active principles, at least one compound that stimulates insulin secretion and at least one compound of the formula (I), in combination with one or more pharmaceutically acceptable excipients.

This composition is particularly suitable for treating diabetes, more particularly non-insulin-dependent diabetes. It is particularly suitable for reducing hyperglycaemia in non-insulin-dependent diabetes.

The compound of the formula (I) is defined in the following manner:

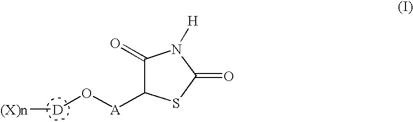

in which A represents a saturated or unsaturated, linear or branched hydrocarbon-based group containing from 2 to 16 carbon atoms,

D represents a homo-carbon-based or hetero-carbon-based, mono-, bi- or tricyclic aromatic structure, possibly including one or more hetero atoms,

X represents a substituent of the aromatic structure, chosen from hydrogen, an alkyl group containing from 1 to 6 carbon atoms, an alkoxy group containing from 1 to 6 carbon atoms, an alkoxyalkyl group in which the alkoxy and alkyl groups are defined as above, an aryl group defined as an aromatic cyclic structure comprising one or two rings optionally including one or two hetero atoms in the ring, such as, for example, a phenyl or an α- or β-naphthyl, an arylalkyl group in which the alkyl group is defined as above and the aryl group is defined as above and optionally comprises one or more substituents, an arylalkylaryl group in which the arylalkyl and aryl fractions are defined as above, a halogen, a trifluoromethyl, a cyano, a hydroxyl, a nitro, an amino, a carboxyl, an alkoxycarbonyl, a carboxamide, a sulphonyl, a sulphone, a sulphonamide, a sulphamoyl, an alkylsulphonylamino, an acylamino and a trifluoromethoxy,

n is an integer ranging from 1 to 3, with the restriction that if A represents a butyl radical,

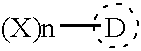

does not represent a 4-chlorophenyl group.

In the text hereinabove, among the aromatic radicals D, homo-carbon-based structures that may be mentioned include the phenyl, α-naphthyl, β-naphthyl, anthracenyl and fluorenyl radicals. Among the heterocyclic aromatic radicals that may be mentioned are pyridyl and the quinolyl or carbazolyl ring.

D preferably represents a phenyl or naphthyl radical.

Among the alkyl groups containing from 1 to 6 carbon atoms that may especially be mentioned are the methyl, ethyl, propyl, isopropyl, butyl, isobutyl, tert-butyl, pentyl and hexyl radicals. Among the alkoxy groups containing from 1 to 6 carbon atoms that may especially be mentioned are the methoxy, ethoxy, propoxy, isopropoxy, butoxy and isobutoxy radicals. Among the halogen groups that may especially be mentioned are fluorine, chlorine, bromine and iodine.

The A chain is a linear or branched hydrocarbon-based chain containing from 2 to 16 carbon atoms, that is saturated or contains one or more ethylenic groups, optionally substituted with at least one hydroxyl radical or with a phenyl radical. Examples of linear alkyl radicals that may especially be mentioned include the divalent ethyl, propyl, butyl, pentyl, hexyl, octyl, nonyl, decyl, dodecyl or hexadecyl radical. Among the branched alkyl chains that may especially be mentioned are the divalent 2-ethylhexyl, 2-methylbutyl, 2-methylpentyl, 1-methylhexyl and 3-methylheptyl radicals. Among the monohydroxyalkyl chains that are preferred are radicals containing 2 or 3 carbon atoms, such as 2-hydroxyethyl, 2-hydroxypropyl or 3-hydroxypropyl. Among the polyhydroxyalkyl chains that are preferred are radicals containing 3 to 6 carbon atoms and 2 to 5 hydroxyl radicals, such as 2,3-dihydroxypropyl, 2,3,4-trihydroxybutyl or 2,3,4,5-tetrahydroxypentyl or a pentaerythritol residue. Among the hydrocarbon-based chains containing from 2 to 16 carbon atoms and one or more ethylenic groups, mention may be made especially of the divalent allyl radical.

The divalent ethyl or propyl radical is preferred.

The present invention relates also to the tautomeric forms of the compounds of the general formula (I), to the enantiomers, diastereoisomers and epimers of these compounds, and also to the solvates thereof.

It may be conceived that the ketone functions borne by the thiazolidine ring can enolize and give rise to mono-enols.

The thiazolidinedione derivatives may, in this case, be salified and be in the form of basic salts.

Examples of basic salts of the compounds of the general formula (I) include pharmacologically acceptable salts, such as sodium salts, potassium salts, magnesium salts, calcium salts, amine salts and other salts of the same type (aluminium, iron, bismuth, etc.). The amine salts that are not pharmacologically acceptable may serve as a means of identification, purification or resolution.

Among the compounds of the general formula (I) according to the invention, mention will be made more particularly, as compounds that are currently preferred, of:

5-[3-(4-fluorophenoxy)propyl]thiazolidine-2,4-dione

5-(2-phenoxyethyl)thiazolidine-2,4-dione

5-[2-(4-fluorophenoxy)ethyl]thiazolidine-2,4-dione

5-{[1-hydroxy-2-(4-fluorophenoxy)]ethyl}thiazolidine-2,4-dione

5-{[2-hydroxy-3-(4-fluorophenoxy)]propyl}thiazolidine-2,4-dione

5-[1-methyl-2-phenoxyethyl]thiazolidine-2,4-dione

5-[2-(4-cyanophenoxy)ethyl)]thiazolidine-2,4-dione

5-[2-(2-fluorophenoxy)ethyl]thiazolidine-2,4-dione

5-[2-(2-naphthyloxy)ethyl]thiazolidine-2,4-dione

and pharmacologically acceptable salts thereof.

These compounds have been described in patent application WO 97/47612.

It is preferred to use 5-[2-(4-cyanophenoxy)ethyl)]thiazolidine-2,4-dione.

The expression “compound that stimulates insulin secretion” means a compound that stimulates insulin secretion by the pancreatic β cells. The compounds that stimulate insulin secretion by the pancreatic β cells are, more specifically, compounds that act on specific receptors located especially at the surface of the pancreatic β cells.

According to the invention, the compound that stimulates insulin secretion is preferably chosen from sulphonylureas.

Examples of sulphonylureas that may especially be mentioned include acetohexamide, carbutamide, gliquidone, glisentide, glisolamide, glisoxepide, glycyclamide, glibomuride, chlorpropamide, tolazamide, tolbutamide, tolcyclamide, glipizide, gliclazide, glimepiride and glibenclamide.

Glipizide, gliclazide, glimepiride or glibenclamide is preferably used.

Repaglinide, nateglinide or mitiglinide may also be mentioned as compounds that stimulate insulin secretion.

The compositions of the invention contain therapeutically effective amounts of the various active principles. The ratios of the respective amounts of compound that stimulates insulin secretion and of compound of the formula (I) thus vary in consequence.

The weight ratio of compound that stimulates insulin secretion to the compound of the formula (I) preferably ranges from 10⁻³ to 40, preferably from 10⁻³ to 10 and better still from 10⁻³ to 1.

The compositions of the invention are preferably administered parenterally, or better still orally, although other routes of administration, such as, for example, rectal administration, are not excluded.

When oral administration is envisaged, the compositions of the invention are in the form of gel capsules, effervescence tablets, coated or uncoated tablets, sachets, sugar-coated tablets, drinkable vials or solutions, microgranules or sustained-release forms.

When parenteral administration is envisaged, the compositions of the invention are in the form of injectable solutions and suspensions packaged in vials or bottles for slow venous infusion.

The forms for oral administration are prepared by mixing the active substance with various types of excipients or vehicles, such as fillers, disintegration (or crumbling) agents, binders, dyes, flavour enhancers and the like, followed by shaping the mixture.

The dye can be any dye authorized for pharmaceutical use.

Examples of flavour enhancers include cocoa powder, mint, borneol and cinnamon powder.

Examples of binders that may be mentioned are polyvinylpyrrolidone, hydroxypropylmethylcellulose, alginic acid, carbomer, carboxymethylcellulose, dextrin, ethylcellulose, starch, sodium alginate, polymethacrylate, maltodextrin, liquid glucose, magnesium aluminium silicate, hydroxyethylcellulose, hydroxypropylcellulose, ethylcellulose, methylcellulose and guar gum.

It is possible to use alginic acid, sodium carboxymethylcellulose, colloidal silicon dioxide, sodium croscarmellose, crospovidone, guar gum, magnesium aluminium silicate, methylcellulose, microcrystalline cellulose, potassium polacrilin, cellulose powder, pregelatinized starch, sodium alginate or sodium starch glycolate as disintegration agent.

The fillers are, for example, cellulose, lactose, calcium hydrogen phosphate and microcrystalline cellulose.

The tablets can be obtained in a conventional manner by compressing granules in the presence of one or more lubricants. Suitable lubricants are calcium stearate, glyceryl monostearate, glyceryl palmitostearate, hydrogenated castor oil, hydrogenated plant oil, light mineral oil, magnesium stearate, polyethylene glycol, sodium benzoate, sodium lauryl sulphate, stearyl sodium fumarate, stearic acid, talc and zinc stearate. These tablets can then be coated using polymers in solution or suspension, such as hydroxypropylmethylcellulose or ethylcellulose.

The granules used to do this are prepared, for example, by using the wet granulation process starting with a mixture of the active principles with one or more excipients such as a binder, a crumbling agent (or disintegration agent) and a filler.

In order to obtain hard capsules, the mixture of active principles with a suitable filler (for example lactose) is incorporated into empty gelatine capsules, optionally in the presence of a lubricant such as magnesium stearate, stearic acid, talc or zinc stearate.

Gel capsules or soft capsules are prepared by dissolving the active principles in a suitable solvent (for example polyethylene glycol), followed by incorporation into soft capsules.

The forms for parenteral administration are obtained in a conventional manner by mixing the active principles with buffers, stabilizers, preserving agents, solubilizing agents, tonicity agents and suspension agents. In accordance with the known techniques, these mixtures are subsequently sterilized and then packaged in the form of intravenous injections.

As buffer, a person skilled in the art can use buffers based on organophosphate salts.

Examples of suspension agents include methylcellulose, hydroxyethylcellulose, hydroxypropylcellulose, acacia and sodium carboxymethylcellulose.

Examples of solubilizing agents include castor oil solidified with polyoxyethylene, polysorbate 80, nicotinamide or macrogol.

In addition, stabilizers that are useful according to the invention are sodium sulphite and sodium metasulphite, while mention may be made of sodium p-hydroxybenzoate, sorbic acid, cresol and chlorocresol as preserving agents. For the preparation of an oral solution or suspension, the active principles are dissolved or suspended in a suitable vehicle with a dispersant, a wetting agent, a suspension agent (for example polyvinylpyrrolidone), a preserving agent (such as methylparaben or propylparaben), a flavour enhancer or a dye.

For the preparation of suppositories, the active principles are mixed in a manner that is known per se with a suitable base constituent, such as polyethylene glycol or semi-synthetic glycerides.

For the preparation of microcapsules, the active principles are combined with suitable diluents, suitable stabilizers, agents that promote the sustained release of the active substances or any other type of additive for the formation of a central core which is then coated with a suitable polymer (for example a water-soluble resin or a water-insoluble resin). The techniques known to those skilled in the art will be used for this purpose.

The microcapsules thus obtained are then optionally formulated in suitable dosage units.

The present invention also relates to the use of a compound that stimulates insulin secretion in combination with a compound of the formula (I) as defined above, for the preparation of a medicinal combination for treating diabetes, more particularly non-insulin-dependent diabetes.

According to another of its aspects, the invention relates to the use of a compound that stimulates insulin secretion in combination with the said compound of the formula (I), for the preparation of a medicinal combination for reducing the hyperglycaemia of non-insulin-dependent diabetes.

The present invention also relates to a process for treating diabetes, more particularly non-insulin-dependent diabetes, in a mammal, comprising the administration to the said mammal of the composition according to the present invention.

When the compound that stimulates insulin secretion and the compound of the formula (I) are incorporated into the same unit dose, the unit dose preferably comprises from 1 mg to 2 g of compound that stimulates insulin secretion (the dose depends especially on the active agents under consideration). When the compound that stimulates insulin secretion is chosen from gliclazide, glipizide, glimepiride, glibenclamide, repaglinide, nateglinide and mitiglinide, the unit dose preferably comprises from 1 mg to 300 mg of compound that stimulates insulin secretion.

In this case, the unit dose advantageously comprises from 12.5 to 50 mg of compound of the formula (I) (the dose depending especially on the active agents under consideration).

Naturally, the dosage depends on the active agent under consideration, the mode of administration, the therapeutic indication and the age and state of the patient.

The daily dosage ranges particularly between 2 mg and 4 g of compound that stimulates insulin secretion and between 25 and 100 mg of compound of the formula (I).

Concrete but non-limiting examples of the invention will now be presented. The percentages given are expressed on a weight basis, except where otherwise mentioned.

### EXAMPLE 1

A tablet having the following composition is prepared:

| | | | |
|---|---|---|---|
| 4-[2-(2,4-Dioxothiazolidin-5-yl)- | 50 | mg | 64.9% |
| ethoxy]benzonitrile* | | | |
| Glibenclamide | 2.5 | mg | 3.2% |
| Fine lactose powder | 18 | mg | 23.4% |
| Hydroxypropylcellulose | 2 | mg | 2.6% |
| Sodium croscarmellose | 4 | mg | 5.2% |
| Magnesium stearate | 0.5 | mg | 0.6% |

*also known as 5-[2-(4-cyanophenoxy)ethyl)]thiazolidine-2,4-dione

### EXAMPLE 2

A tablet having the following composition is prepared:

| | | | |
|---|---|---|---|
| 4-[2-(2,4-Dioxothiazolidin-5-yl)- | 50 | mg | 64.9% |
| ethoxy]benzonitrile | | | |
| Glibenclamide | 5 | mg | 6.5% |
| Fine lactose powder | 15.5 | mg | 20.1% |
| Hydroxypropylcellulose | 2 | mg | 2.6% |
| Sodium croscarmellose | 4 | mg | 5.2% |
| Magnesium stearate | 0.5 | mg | 0.6% |

### EXAMPLE 3

A tablet having the following composition is prepared:

| | | | |
|---|---|---|---|
| 4-[2-(2,4-Dioxothiazolidin-5-yl)- | 100 | mg | 73.0% |
| ethoxy]benzonitrile | | | |
| Glibenclamide | 2.5 | mg | 1.8% |
| Fine lactose powder | 22.5 | mg | 16.4% |
| Hydroxypropylcellulose | 4 | mg | 2.9% |
| Sodium croscarmellose | 7 | mg | 5.1% |
| Magnesium stearate | 1 | mg | 0.7% |

### EXAMPLE 4

A tablet having the following composition is prepared:

| | | | |
|---|---|---|---|
| 4-[2-(2,4-Dioxothiazolidin-5-yl)- | 100 | mg | 73.0% |
| ethoxy]benzonitrile | | | |
| Glibenclamide | 5 | mg | 3.6% |
| Fine lactose powder | 20 | mg | 14.6% |
| Hydroxypropylcellulose | 4 | mg | 2.9% |
| Sodium croscarmellose | 7 | mg | 5.1% |
| Magnesium stearate | 1 | mg | 0.7% |

### EXAMPLE 5

A tablet having the following composition is prepared:

| | | | |
|---|---|---|---|
| 4-[2-(2,4-Dioxothiazolidin-5-yl)- | 200 | mg | 80% |
| ethoxy]benzonitrile | | | |
| Glibenclamide | 2.5 | mg | 1.0% |
| Fine lactose powder | 27.5 | mg | 11.0% |
| Hydroxypropylcellulose | 6.5 | mg | 2.6% |
| Sodium croscarmellose | 12 | mg | 4.8% |
| Magnesium stearate | 1.5 | mg | 0.6% |

### EXAMPLE 6

A tablet having the composition below is prepared:

| | | | |
|---|---|---|---|
| 4-[2-(2,4-Dioxothiazolidin-5-yl)- | 200 | mg | 80% |
| ethoxy]benzonitrile | | | |
| Glibenclamide | 5 | mg | 2.0% |
| Fine lactose powder | 25 | mg | 10.0% |
| Hydroxypropylcellulose | 6.5 | mg | 2.6% |
| Sodium croscarmellose | 12 | mg | 4.8% |
| Magnesium stearate | 1.5 | mg | 0.6% |

In the following data show the synergetic effect for the association of 4-[2-(2,4-dioxothiazolidin-5-yl)ethoxy]benzonitrile and glibenclamide for the insulogenic index (sensibility of β cells) and not for the two compounds taken separately.

Combination of (4-[2-(2,4-dioxothiazolidin-5-yl)ethoxy]benzonitrile) and Glibenclamide: Investigation of a Beneficial Effect of this Combination on the Treatment of Hyperglycaemia

Protocol

Diabetes is induced in male rats (source: Iffa Credo) by intravenous administration of streptozotocin on the day of birth.

This model of diabetic rats, n0STZ, is characterized by moderate hyperglycaemia, glucose intolerance and impaired insulin response to glucose, but shows no insulin resistance.

Method

The rats are selected according to the criterion of hyperglycaemia 170±15 mg/dl.

The animals are grouped into 5 rats per batch and divided into 4 batches

1 control batch

1 4-[2-(2,4-dioxothiazolidin-5-yl)ethoxy]benzonitrile batch

1 glibenclamide batch

1 batch combining glibenclamide+4-[2-(2,4-dioxothiazolidin-5-yl)-ethoxy]benzonitrile

The products are administered 2 hours before the glucose tolerance test, at a rate of 12.5 mg/kg for 4-[2-(2,4-dioxothiazolidin-5-yl)ethoxy]benzonitrile, 2 mg/kg for glibenclamide and the combination of the 2 products at the doses used in monotherapy.

The glucose tolerance test (OGTT) is performed on the conscious animal and consists in administering glucose at a rate of 2 g/kg. The plasmatic glucose clearance is analysed after this glucose load, over 2 hours (0-10-20-30-45-60-90 and 120 min).

In addition to plasmatic glycaemia, insulinaemia is also determined.

Results—Table 1

Acute administration of 12.5 mg/kg of 4-[2-(2,4-dioxothiazolidin-5-yl)ethoxy]benzonitrile or 2 mg/kg of glibenclamide has little or no effect on carbohydrate tolerance.

A 6% decrease is observed in the case of 4-[2-(2,4-dioxothiazolidin-5-yl)ethoxy]benzonitrile and a 6% increase in the case of glibenclamide (curves 1 and 2).

The combination of 4-[2-(2,4-dioxothiazolidin-5-yl)ethoxy]benzonitrile and glibenclamide shows an improvement in glucose tolerance (−15%) and insulinogenic index* (+23%) (histogram 1). The latter effect, namely an improvement in the glucose sensitivity of the beta cells, induced by the combination, was not foreseeable. * Insulinogenic index=ΔI/ΔG with ΔI=variation in the plasmatic insulin level ΔG=variation in the plasmatic glucose

Conclusion

The combination of 4-[2-(2,4-dioxothiazolidin-5-yl)ethoxy]benzonitrile and glibenclamide used in monotherapy at doses inducing little or no antidiabetic activity generates a synergistic effect allowing an improvement in carbohydrate tolerance.

[image]

[image]

[image]

Effect of the Combination of 4-[2-(2,4-dioxothiazolidin-5-yl)ethoxy]benzonitrile (Compound A)+Glibenclamide

TABLE OF RESULTS
Plasmatic glucose (mg/dl)

| | 0 | 10 | 20 | 30 | 45 | 60 | 90 | 120 | AUC |
|---|---|---|---|---|---|---|---|---|---|
| Control n0STZ | 167 | 330 | 421 | 462 | 472 | 498 | 454 | 393 | 51920 |
| sem | 10 | 23 | 44 | 54 | 70 | 79 | 75 | 65 | 7447.5 |
| Compound A | 160 | 321 | 381 | 418 | 424 | 447 | 419 | 378 | 47702.5 |
| sem | 8 | 4 | 15 | 13 | 12 | 24 | 25 | 32 | 2342.5 |
| Glibenclamide | 138 | 312 | 407 | 470 | 520 | 513 | 507 | 448 | 55027.5 |
| sem | 7 | 29 | 37 | 48 | 49 | 42 | 59 | 66 | 5735 |
| Combination | 140 | 271 | 347 | 377 | 410 | 426 | 384 | 337 | 43902.5 |
| sem | 7 | 3 | 16 | 28 | 43 | 42 | 36 | 31 | 3710 |

Plasmatic insulin (μU/ml)

| | 0 | 10 | 20 | 30 | 45 | 60 | 90 | 120 | AUC |
|---|---|---|---|---|---|---|---|---|---|
| Control n0STZ | 50.6 | 94.8 | 111.9 | 106.1 | 84.3 | 78 | 90.9 | 94.7 | 10813.25 |
| sem | 12 | 18 | 21 | 25 | 16 | 13 | 10 | 11 | 1760 |
| Compound A | 47.2 | 100.8 | 89.6 | 84.5 | 74.6 | 77 | 93.8 | 101.5 | 10384.25 |
| sem | 5 | 17 | 14 | 15 | 9 | 10 | 12 | 19 | 1527.5 |
| Glibenclamide | 84.2 | 116.9 | 129.4 | 105.8 | 100 | 88.3 | 92.5 | 97.5 | 11930.75 |
| sem | 4 | 16 | 12 | 4 | 11 | 16 | 12 | 10 | 1385 |
| Combination | 88.7 | 104.6 | 100.2 | 86 | 97.4 | 90.4 | 92.1 | 99.1 | 11311 |
| sem | 15 | 15 | 12 | 15 | 19 | 14 | 7 | 8 | 1462.5 |

5 rats/batch:
4-[2-(2.4-dioxothiazolidin-5-yl)ethoxy]benzonitrile (compound A) 12.5 mg/kg
Glibenclamide 2 mg/kg
Combination of 4-[2-(2.4-dioxothiazolidin-5-yl)ethoxy]benzonitrile (compound A) (12.5 mg/kg) + glibenclamide (2 mg/kg)

1. Pharmaceutical composition comprising, as active principles, (i) at least one compound that stimulates insulin secretion and (ii) at least one compound of the formula (I) in combination with one or more pharmaceutically acceptable excipients, the compound of the formula (I) being defined in the following manner: 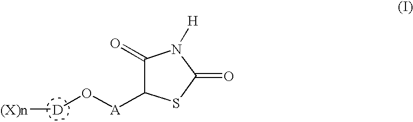
in which A represents a saturated or unsaturated, linear or branched hydrocarbon-based group containing from 2 to 16 carbon atoms,
D represents a homo-carbon-based or hetero-carbon-based, mono-, bi- or tricyclic aromatic structure possibly including one or more hetero atoms,
X represents a substituent of the aromatic structure, chosen from hydrogen, an alkyl group containing from 1 to 6 carbon atoms, an alkoxy group containing from 1 to 6 carbon atoms, an alkoxyalkyl group in which the alkoxy and alkyl groups are defined as above, an aryl group defined as an aromatic cyclic structure comprising one or two rings optionally including one or two hetero atoms in the ring, such as, for example, a phenyl or an α- or β-naphthyl, an arylalkyl group in which the alkyl group is defined as above and the aryl group is defined as above and optionally comprises one or more substituents, an arylalkylaryl group in which the arylalkyl and aryl fractions are defined as above, a halogen, a trifluoromethyl, a cyano, a hydroxyl, a nitro, an amino, a carboxyl, an alkoxycarbonyl, a carboxamide, a sulphonyl, a sulphone, a sulphonamide, a sulphamoyl, an alkylsulphonylamino, an acylamino and a trifluoromethoxy, 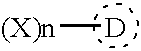
n is an integer ranging from 1 to 3, with the restriction that if A represents a butyl radical, [lacuna] does not represent a 4-chlorophenyl group:

2. Composition according to claim 1, for treating diabetes.

3. Composition according to either of claims 1 and 2, for treating non-insulin-dependent diabetes.

4. Pharmaceutical composition according to any one of claims 1 to 3, characterized in that the weight ratio of the compound that stimulates insulin secretion to the compound of the formula (I) ranges from 10⁻³ to 40, preferably from 10⁻³ to 10 and better still from 10⁻³ to 1.

5. Pharmaceutical composition according to any one of the preceding claims, characterized in that the compound that stimulates insulin secretion is chosen from sulphonylureas.

6. Pharmaceutical composition according to the preceding claim, characterized in that the compound that stimulates insulin secretion is chosen from acetohexamide, carbutamide, gliquidone, glisentide, glisolamide, glisoxepide, glycyclamide, glipizide, gliclazide, glimepiride, glibenclamide, glibornuride, chlorpropamide, tolazamide, tolbutamide and tolcyclamide.

7. Composition according to any one of the preceding claims, characterized in that the compound of the formula (I) is chosen from:
5-[3-(4-fluorophenoxy)propyl]thiazolidine-2,4-dione
5-(2-phenoxyethyl)thiazolidine-2,4-dione
5-[2-(4-fluorophenoxy)ethyl]thiazolidine-2,4-dione
5-{[1-hydroxy-2-(4-fluorophenoxy)]ethyl}thiazolidine-2,4-dione
5-{[2-hydroxy-3-(4-fluorophenoxy)]propyl}thiazolidine-2,4-dione
5-[1-methyl-2-phenoxyethyl]thiazolidine-2,4-dione
5-[2-(4-cyanophenoxy)ethyl)]thiazolidine-2,4-dione
5-[2-(2-fluorophenoxy)ethyl]thiazolidine-2,4-dione
5-(2-(2-naphthyloxy)ethyl]thiazolidine-2,4-dione
and pharmacologically acceptable salts thereof.

8. Composition according to claim 7, characterized in that the compound of the formula I is 5-[2-(4-cyanophenoxy)ethyl)]thiazolidine-2,4-dione.

9. Composition according to any one of the preceding claims, that is suitable for oral administration.

10. Use of a compound that stimulates insulin secretion in combination with a compound of the formula (I) as defined in claim 1, for the preparation of a medicinal combination for treating diabetes.

11. Use according to claim 10, for the preparation of a medicinal combination for treating non-insulin-dependent diabetes.

12. Use according to either of claims 10 and 11, characterized in that the compound that stimulates insulin secretion is chosen from sulphonylureas.

13. Use according to the preceding claim, characterized in that the compound that stimulates insulin secretion is chosen from glipizide, gliclazide, glimepiride, glibenclamide, glibornuride, chlorpropamide, tolazamide, tolbutamide and tolcyclamide.

14. Use according to one of claims 10 to 13, characterized in that the compound of the formula (I) is chosen from:
5-[3-(4-fluorophenoxy)propyl]thiazolidine-2,4-dione
5-(2-phenoxyethyl)thiazolidine-2,4-dione
5-[2-(4-fluorophenoxy)ethyl]thiazolidine-2,4-dione
5-{[1-hydroxy-2-(4-fluorophenoxy)]ethyl}thiazolidine-2,4-dione
5-{[2-hydroxy-3-(4-fluorophenoxy)]propyl}thiazolidine-2,4-dione
5-[1-methyl-2-phenoxyethyl]thiazolidine-2,4-dione
5-[2-(4-cyanophenoxy)ethyl)]thiazolidine-2,4-dione
5-[2-(2-fluorophenoxy)ethyl]thiazolidine-2,4-dione
5-[2-(2-naphthyloxy)ethyl]thiazolidine-2,4-dione
and pharmacologically acceptable salts thereof.

15. Use according to any one of claims 10 to 14, characterized in that the medicinal combination is in the form of a unit dose comprising a compound that stimulates insulin secretion and a compound of the formula (I).

16. Use according to the preceding claim, characterized in that the unit dose comprises from 1 mg to 2 g of a compound that stimulates insulin secretion and from 12.5 to 50 mg of a compound of the formula (I).
